(19) United States
(12) Patent Application Publication
(10) Pub. No.: US 2007/0010807 A1
(43) Pub. Date: Jan. 11, 2007

(54) Apparatus and methods for modulating the size of an implantable sling

(75) Inventor: Michael S. H. Chu, Brookline, MA (US)

Correspondence Address: FISH & NEAVE IP GROUP;ROPES & GRAY LLP, ONE INTERNATIONAL PLACE, BOSTON, MA, 02110-2624, US

(73) Assignee: Boston Scientific Scimed, Inc.

(21) Appl. No.: 11/178,964

(22) Filed: Jul. 11, 2005

(51) Int. Cl. A61B 17/32; A61B 18/18; A61B 17/08

(52) U.S. Cl. 606/39; 606/151; 606/167

(57) ABSTRACT

The invention provides methods and devices for modulating the size of an implantable sling.

### FIELD OF THE INVENTION

The invention generally relates to devices and methods for customizing an implantable, supportive sling. More specifically, in various illustrative embodiments, the invention provides devices and methods for cutting and/or fusing sections of an implantable sling to modulate the size of the implantable sling.

### BACKGROUND

Anatomical tissues may become weakened or damaged by age, injury, or disease. This decrease in the structural integrity of anatomical tissues may have significant medical consequences. Even in the absence of tissue necrosis, weakening of an anatomical structure may impair one or more of the biological functions of the tissue. To help alleviate this impact on biological function, implantable, supportive slings have been developed. These slings can be implanted into a patient to provide support for the weakened or damaged tissue. The support provided by the sling mimics the natural position and structure of the tissue, and thereby helps decrease or eliminate impairment of biological function resulting from tissue weakening or damage. Although supportive slings have been used in numerous contexts to address the weakening of a variety of anatomical tissues, they have proven particularly useful for decreasing urinary incontinence resulting from weakening or damage to urethral, periurethral and/or bladder tissue.

Stress urinary incontinence (SUI) affects primarily women, but also men, and is generally caused by two conditions, intrinsic sphincter deficiency (ISD) and hypermobility. These conditions may occur independently or in combination. In ISD, the urinary sphincter valve, located within the urethra, fails to close properly (coapt), causing urine to leak out of the urethra during stressful activity. Hypermobility is a condition in which the pelvis floor is distended, weakened, or damaged, causing the bladder neck and proximal urethra to rotate and descend in response to increases in intra-abdominal pressure (e.g., due to sneezing, coughing, straining, etc.). As a result, the patient's response time becomes insufficient to promote urethral closure and, consequently, the patient suffers from urine leakage and/or flow. SUI has a variety of causes including, but not limited to, pregnancy, aging, infection, injury, congenital defect, and disease.

A popular treatment of SUI involves placement of implantable slings under the bladder neck or the mid-urethra to provide a urethral platform. Placement of the sling limits the endopelvis fascia drop. There are various methods for placing the sling. Slings can be affixed and stabilized using traditional bone anchoring approaches, as well as recently developed anchor-less methods. Additionally, a variety of implantation procedures, including various routes of administration, exist. These procedures provide physicians with a range of implantation options. Physicians can readily select amongst the various implantation procedures based on numerous patient-specific factors including, but not limited to, age, gender, overall health, location of tissue defect, the degree of tissue impairment, and the like. Furthermore, physicians can select from amongst numerous sling delivery devices that facilitate sling placement.

Despite the numerous advances in sling design, implantation methodologies, and delivery devices, a significant limitation remains unaddressed. Implantable slings are manufactured in various standard sizes and shapes. However, patient size varies significantly across the population. Furthermore, the degree of sling tension required to provide adequate tissue support varies across patients depending on numerous factors including the location and extent of the injury or defect. As a result of these and other patient specific factors, there exists a need for methods and devices to modulate the size of an implantable sling.

### SUMMARY OF THE INVENTION

The invention addresses the deficiencies of the prior art by providing, in various embodiments, methods and devices for modulating the size of an implantable sling. Specifically, the invention provides methods and devices for cutting through and/or fusing sections of an implantable sling to modulate the size of the sling. Such methods and devices can be used to customize slings for use in particular patients.

As described above, implantable slings can be used to provide support to any of a number of diverse anatomical tissues. Physicians may select from amongst any of a number of procedures for implanting and securing a particular sling in a patient. These procedures may involve different routes of administration, and thus may require varying lengths of sling to achieve the desired degree of support. Furthermore, patient size, both among genders and across genders, may vary significantly. Given these and other variables, methods and devices for customizing the size of an implantable sling, thereby achieving a therapeutically desirable fit in each patient, are of substantial value.

In general, the invention is directed to methods and devices for modulating the length of an implantable sling. The length of an implantable sling can be modulated by, for example, (i) cutting through a sling, (ii) cutting through a sling and concurrently fusing the two resulting portions together, (iii) fusing two portions of a single continuous sling together, or (iv) fusing separate and unconnected sling sections together without the need for any cutting. The invention contemplates that the length of the sling can be modulated by cutting through and/or fusing the sling along a free end, or along a portion of the sling that is not a free end of the sling (e.g., fusing a looped section). In certain embodiments, one or both of the free-ends of a sling have been manufactured to contain one or more useful features, such as, without limitation, a tissue dilator and/or a mechanism for associating the sling with a delivery device. When a free-end of a sling contains one or more useful features, it may be advantageous to modify the size of the sling by cutting through and/or fusing the sling at a portion of the sling other than a free end.

In one embodiment, the implantable sling is sized and shaped for providing urethral support. Exemplary slings, whether urethral slings or other supportive slings, can be made from any of a variety of materials. Such materials include, without limitation, nylon, polyethylene, polyester, polypropylene, fluoropolymers, polyglycolic acid, polylactic acid, copolymers thereof, combinations thereof, or other suitable synthetic material(s). In some embodiments, a supportive sling is derived, in whole or in part, from mammalian tissue(s) or a combination of mammalian tissue(s) and synthetic material(s). Preferably the sling is heat responsive. Optionally, the sling materials may incorporate or be treated with one or more agents. Exemplary agents include, without limitation, agents that provide a therapeutic effect, such as reducing discomfort, reducing inflammation, reducing the likelihood of infection, and/or promoting tissue growth.

In a first aspect, the invention provides methods and devices for modulating the length of an implantable sling. An exemplary device according to this aspect of the invention includes a handle, a heating element extending distally from and in interoperable interconnection with the distal portion of the handle, and a sling guide housing. The sling guide housing helps guide a portion of the implantable sling into contact with the heating element. According to one feature, contacting a surface of the sling with the heating element cuts through the sling. According to another feature, contacting the heating element with two sling sections fuses the two sling sections together at the point of contact. According to one implementation, a looped portion of a sling may be brought into contact with the heating element to fuse two legs of the loop together to shorten the length of the sling.

In one embodiment, the device includes a battery compartment. The battery compartment is sized and shaped for housing one or more batteries for supplying power to the heating element. Optionally, the battery compartment is contained within the handle. In another embodiment, the device includes a power supply for operable interconnection with an external power source. The power supply may be rechargeable or only able to supply power to the heating element when operatively connected to the external power source.

Regardless of whether the device includes a battery compartment, a power supply compatible with an external power source, or some alternative power supply, the device may also include a switch for modulating the power from the power supply to the heating element. The switch may be in any configuration, preferably, a configuration that facilitates use of the switch by the medical operator using the device. Exemplary switches include switches with two positions, an “off”and an “on” position. Further exemplary switches include more than two positions, for example, three positions. A three position switch may, in one embodiment, include an “off” position, a low “on” position, and a high “on” position. The two “on” positions may indicate varying levels of power supplied from the power source to the heating element to regulate the temperature of the heating element. The switch may also include more discrete positions or be of a continuous nature to provide a medical operator with finer control over the temperature of the heating element. In any of the foregoing, the switch may be moved between positions by sliding, turning, rotating, pressing, flipping, or otherwise manipulating the switch.

In another embodiment, the device includes a temperature control. This control element enables the medical operator to select a particular heating element temperature. When the device includes a temperature control element, it may be incorporated into the switch or it may be separate from the switch.

According to one configuration, the handle and the sling guide housing are aligned along the same axis. However, in other configurations, the handle and the sling guide housing are aligned along different axes, for example, to form a gun-shaped device. According to another feature, the sling guide housing and the handle may pivot relative to each other and may be arranged at any suitable angle relative to each other.

Regardless of the angle of the handle relative to the sling guide housing, the invention contemplates that the sling guide housing may extend and retract axially. Movement of the sling guide housing permits regulation of the axial distance between the heating element and a distal-most end of the sling guide housing. Furthermore, movement of the sling guide housing permits regulation of the axial distance between a distal end of the handle and the proximal-most end of the sling guide housing.

As mentioned above, the sling guide housing helps guide an implantable sling into contact with the heating element. Optionally, the sling guide housing may also help shield the heating element. Shielding of the heating element can help prevent injury to the device operator, and can also help prevent inadvertent contact between the heating element and patient tissue. Shielding may be particularly important for embodiments of the invention in which the device is used concomitantly with sling implantation.

In one embodiment, the sling guide housing includes one or more slots. According to one configuration, one or more slots are axially oriented and extend radially through the sling guide housing. According to one implementation, slots help guide a portion of a sling or multiple sling portions through the housing and into contact with the heating element. In a further configuration, the one or more slots also extend from a distal-most end of the sling guide housing proximally toward the handle to form an axially oriented slot at the distal end extending radially through a wall of the sling guide housing. In some embodiments, the sling guide housing includes two axially oriented slots diametrically opposed to each other. These slots may be of the same or of different widths. For example, the width of the first slot may be greater than the width of the second slot. The slots, whether of the same or of different widths, can be used to help guide a portion of the sling into and out of the sling guide housing, and furthermore to help guide a portion of the sling into contact with the heating element.

In another embodiment, the housing includes a radially (e.g., substantially normal to a longitudinal axis of the housing) oriented, radially extending notch near the distal end of the housing. In one configuration, the radially extending notch forms a space, not only between a proximal portion of the housing and a distal portion of the housing, but also between the heating element and an inner surface of the distal portion of the housing.

In any of the foregoing, the sling guide housing may be made of any suitable biocompatible material, for example, a biocompatible polymeric material. In certain embodiments, all or a portion of the sling guide housing may be made from substantially transparent or translucent material to enable a medical operator to view the sling joining or cutting, as the case may be, and to facilitate accurate use of the device by the medical operator.

According to another embodiment, the heating element includes terminals for electro-communication with the power supply. The terminals connect the power supply to the heating element via insulator portions. In one embodiment, the power supply is a battery, and the terminals connect the battery located in a battery compartment to the heating element. In another embodiment, the handle contains the battery compartment, and the heating element is in interoperable electrical interconnection with the battery compartment in the handle via the terminals.

Exemplary heating elements for use with the methods and devices of the invention are sized and shaped to cut through an implantable sling and/or fuse together separate and independent sling sections or a section of a single sling. In one embodiment, the heating element includes at least two sections, and at least one of the sections of the heating element is sized and shaped to cut through and/or fuse an implantable sling material. In such embodiments of the invention, other section(s) of the heating element may or may not contact the sling material. By way of example, other section(s) of the heating element may be sized and shaped to help guide the sling material, but not cut through it. By way of example, the other section(s) of the heating element may either not get hot enough to cut through the sling material or may be sized and shaped for only incidental contact with the sling material. By incidental contact is meant that contact between a portion of the heating element and a portion of the sling material is for a time or at a temperature that is less than the time or temperature sufficient to cut through and/or fuse the sling material. In another embodiment, the heating element includes at least three sections, at least one of which is sized and shaped to cut through the sling material.

The invention contemplates heating elements of varying sizes and shapes. The size and shape of the heating element can be readily altered to modify the devices of the invention for use with particular slings and/or sling sections. In one embodiment, the heating element includes at least (1) a first section including a first leg extending along a first axis and a second leg extending along a second axis and (2) a second section extending from the first section. According to one feature, the first section is substantially V-shaped. Specifically, the first leg extends axially in a distal direction from the insulator portion, and the second leg extends from and at an angle to the first leg. The first leg extending along the first axis and the second leg extending along the second axis are angled relative to each other to form a substantially V-shaped section. The individual legs may be substantially straight or curved. The second section extends radially from the second leg of the first section and is substantially U-shaped. The size and shape of the U-shaped section may vary according to the particular use of the device.

In another embodiment, the heating element includes at least (1) a first section including a first leg extending along a first axis and a second leg extending along a second axis, (2) a second section spaced apart from and of a substantially similar shape as the first section, and (3) a third section radially extending from both the first section and the second section. According to one feature, the first section is substantially V-shaped. Specifically, the first leg extends axially in a distal direction from the insulator portion, and the second leg extends from and at an angle to the first leg. The first leg extending along the first axis and the second leg extending along the second axis are angled relative to each other to form a substantially V-shaped section. The second section, which is spaced apart from and substantially symmetrical to the first section, is of a substantially similar size and shape as the first section. The third section is substantially U-shaped. The U-shaped section extends radially from both the second leg of the first section and the second leg of the second section. The size and shape of the U-shaped section may vary according to the particular use of the device.

According to another embodiment, the heating element includes at least (1) a first section including a substantially straight leg extending along a first axis, (2) a second section spaced apart from and of a substantially similar shape as the first section, and (3) a curved third section extending between the first and second sections. According to one feature, the first section and the second section extend axially in a distal direction from the insulator portions. The curved third section extends distally and radially from the first and second sections, and is sized and shaped such that a portion of the curved section is substantially perpendicular to the first and second sections.

The invention contemplates that the various sections and portions of the heating element may be of varying sizes, shapes, and configurations. In one embodiment, the section of the heating element doing the cutting and/or fusing has a length greater than or equal to the width of the implantable sling. In another embodiment, the cutting and/or fusing section of the heating element has a length less than or equal to the width of the implantable sling.

In any of the foregoing, the invention contemplates that the heating element can be composed of any of a number of heat tolerant materials. For example, all or a portion of the heating element can be composed, in whole or in part, of nickel chromium. Further exemplary materials include, without limitation, other nickel alloys and tungsten. In one embodiment, the heating element is composed of materials that are both heat tolerant and corrosion resistant.

In another aspect, the invention provides methods and devices for fusing a first separate and independent implantable sling section with a second separate and independent (i.e., unattached to the first sling section) implantable sling section. Devices according to this aspect of the invention include a handle, a heating element in interoperative interconnection with the handle, and a sling guide housing. The sling guide housing is used to guide the first sling section and the second sling section into contact with the heating element to fuse the first and second sling sections together. In this aspect of the invention, the handle, sling guide housing, and heating element may include any feature of the previously described embodiments of the invention. Additionally, this aspect of the invention may also include a battery compartment and/or power supply as described above.

According to certain features of this aspect of the invention, the housing includes a radially oriented and radially extending notch. The portion of the housing located proximal to the notch is referred to as the proximal portion of the housing, and the portion of the housing distal to the notch is referred to as the distal portion of the housing. The distal portion of the housing can act as a spacer element during sling placement. By way of example, when a spacer-containing housing is used concomitantly with sling implantation, the outer distal-most surface of the spacer element can be placed in contact with the patient's tissue at the anatomical site to be supported. The width (axial thickness) of the spacer element can be calibrated to provide the appropriate gap for the desired tensioning between the sling and the anatomical site to be supported.

As described above, the housing may be extended and retracted axially. Movement of the housing can be used to modulate the distance between, for example, the heating element and the inner surface of the distal portion of the housing. To fuse two sling sections together, the housing is axially retracted to sandwich the two sling sections between the heating element and the inner surface of the distal portion of the housing.

In another aspect, the invention provides a forceps-like device for modulating the length of an implantable sling. According to one feature, the device can be used to cut through a portion of an implantable sling. According to another feature, the device can be used to fuse two sections of an implantable sling.

An exemplary device according to this aspect of the invention includes a first elongated element and a second elongated element. The first and second elongated elements extend along first and second intersecting axes and cross at a pivot. Each elongated element has, with respect to the pivot, a proximal portion and a distal portion. One or both of the elongated elements may further include a handle portion extending proximally from the proximal portion of the elongated element. Such a handle portion may include, for example, a finger loop and/or a grip. Regardless of the configuration of the handle portion, preferable handle portions facilitate use of the device by a medical operator. In one embodiment, the length of the distal portion of each of the first and second elongated elements is less than the length of the proximal portion of each of the first and second elongated elements (e.g., the elements intersect at a point other than their midpoint). In another embodiment, the length of the distal portion of each of the first and second elongated elements is approximately equal to the length of the proximal portion of each of the first and second elongated elements (e.g., the elements intersect at approximately their midpoint).

The forceps-like device further includes a heating element. The heating element extends along and is in interoperable interconnection with an inner surface of the distal portion of the first elongated element. The heating element is sized and shaped to cut through and/or fuse a portion of an implantable sling. Exemplary heating elements may be configured in any of a number of ways including, but not limited to, substantially extending along a single axis in the plane of the inner surface of the distal portion of the first elongated element. Exemplary heating elements may include a single section extending along a single axis, or may include more than one section extending along and in the plane of the inner surface of the distal portion of the first elongated element. According to certain features, the more than one heating element sections may be substantially parallel to each other. Substantially parallel sections may be continuous (e.g., a ribbon configuration) or physically spaced apart (e.g., non-continuous).

The heating element is in interoperable interconnection with a power supply. In certain embodiments, the device includes a power supply such as, for example, the various power supplies described in detail above. The device may further include a switch or temperature control element. In one embodiment, a portion, for example the proximal portion, of the first elongated element includes the power supply. According to certain features, the power supply may be sized and shaped to serve as a handle portion. Alternatively, the first elongated element may include a handle portion extending proximally from the proximal portion.

The various configurations of the forceps-like device of the invention function in substantially the same manner to cut through a portion of a sling or to fuse two sling sections. In the absence of power from the power supply to the heating element, the forceps-like device is used much like a standard forceps to, for example, manipulate an implantable sling without substantially altering the structure of the sling. According to one feature, in the presence of power to the heating element, the heating element can cut through a portion of an implantable sling to modulate the length of the implantable sling. According to another feature, in the presence of power to the heating element, the heating element can fuse two sling sections to modulate the length of the implantable sling.

Whether the device is used to cut through or fuse an implantable sling, a surface of the implantable sling is brought into contact with the heating element by drawing together the first elongated element and the second elongated element. For example, the distal portion of the first elongated element is brought into contact with the distal portion of the second elongated element. In one embodiment, a portion of an implantable sling is placed between the inner surface of the first elongated element (e.g., the inner surface containing the heating element) and the inner surface of the second elongated element (e.g., the inner surface that does not contain a heating element). According to certain features, the inner surface of the second elongated element may be sized and shaped for positioning the implantable sling with respect to the heating element. According to certain other features, the inner surface of the second elongated element may be sized and shaped to help cut through a portion of a sling or to help fuse two sling sections. Features for positioning or otherwise facilitating sling cutting and/or fusing include, but are not limited to, notches or grooves in the inner surface of the second elongated element.

As mentioned above, the methods and devices of the invention can be used to cut through and/or fuse a sling prior to or concomitantly with a sling implantation procedure. Exemplary methods of concomitant use include concomitant transvaginal use, wherein the sling is implanted transvaginally and customization of sling length occurs transvaginally during the implantation procedure. However, the invention similarly contemplates concomitant use of the methods and devices of the invention during other sling implantation procedures. Concomitant use during some sling implantation procedures necessarily involves insertion of the device of the invention into the patient (e.g., modulation of the sling occurs within the patient's body). Concomitant use during certain other sling implantation procedures does not require such insertion. For implantation procedures where one or more portions of the sling extend out of the patient's body, concomitant use of a device of the invention includes modulating the length of the sling by cutting through a portion of a sling or by fusing two sling sections at a point along the sling that extends outside of the patient's body. In addition to concomitant use, the invention contemplates that sling length can be modified prior to an implantation procedure.

In another aspect, the invention contemplates kits that include a device for cutting through and/or fusing an implantable sling. Exemplary kits include, in addition to a device for cutting through and/or fusing an implantable sling, one or more implantable slings. Additional exemplary kits include a device for cutting through and/or fusing an implantable sling, one or more implantable slings, and one or more sling delivery devices.

According to a feature of any of the above described embodiments, the devices of the invention may be single use disposable devices or may be sterilized for multiple uses.

### BRIEF DESCRIPTION OF THE DRAWINGS

The invention may be better understood by referring to the following description of illustrative embodiments, taken in conjunction with the accompanying drawings, in which like reference designations refer to like components and depicted components are not necessarily drawn to scale.

FIG. 1A depicts a front view of a device for cutting through and/or fusing an implantable sling, according to an illustrative embodiment of the invention.

FIG. 1B depicts a close-up view of a distal portion of the device of FIG. 1A.

FIG. 1C depicts a lateral view of the device of FIG. 1A.

FIGS. 2A-2C depict close-up views of a heating element of the type employed with the device of FIGS. 1A-1C.

FIG. 3 depicts a close-up side view of a distal portion of a device similar to that of FIGS. 1A-1C, except with an alternative heating element configuration for increasing the length of the portion of the sling to be cut and/or fused.

FIG. 4 depicts a close-up side view of a distal portion of a device similar to the device of FIGS. 1A-1C, except with a heating element having a width substantially equal to that of the sling.

FIG. 5 depicts a device for cutting through and/or fusing an implantable sling, and including a sling glide housing with a radially oriented and extending notch, according to another illustrative embodiment of the invention.

FIG. 6 is an enlarged view of a distal end of the handle of the device of FIG. 5, with an illustrative heating element attached.

FIG. 7 depicts a forceps-like device for cutting through and/or fusing an implantable sling, according to another illustrative embodiment of the invention.

FIG. 8 depicts an implantable sling, according to an illustrative embodiment of the invention.

### DESCRIPTION OF ILLUSTRATIVE EMBODIMENTS

As described above in summary, the invention provides, in various illustrative embodiments, methods and devices for modulating the size an implantable sling. The length of an implantable sling can be modulated by, for example, (i) cutting through a sling, (ii) cutting through a sling and concurrently fusing the two resulting portions together, (iii) fusing two portions of a single continuous sling together, or (iv) fusing separate and unconnected sling sections together without the need for any cutting. The invention contemplates that the length of the sling can be modulated by cutting through and/or fusing the sling along a free end, or along a portion of the sling that is not a free end of the sling (e.g., fusing a looped section). In certain embodiments, one or both of the free-ends of a sling have been manufactured to contain one or more useful features, such as, and without limitation, a tissue dilator and/or a mechanism for associating the sling with a delivery device. When a free-end of a sling contains one or more useful features, it may be advantageous to modify the size of the sling by cutting through and/or fusing the sling at a portion of the sling other than a free end. The methods and devices of the invention can be used prior to or concomitantly with an implantation procedure to customize an implantable sling for a particular patient.

Without limitation, examples of slings, sling assemblies, delivery devices and implantation approaches with features that may be employed in illustrative embodiments of the invention are disclosed in U.S. Pat. No. 6,042,534, entitled “Stabilization Sling for Use in Minimally Invasive Pelvic Surgery,” U.S. Pat. No. 6,755,781, entitled “Medical Slings,” U.S. Pat. No. 6,666,817, entitled “Expandable Surgical Implants and Methods of Using Them,” U.S. Pat. No. 6,042,592, entitled “Thin Soft Tissue Surgical Support Mesh,” U.S. Pat. No. 6,375,662, entitled “Thin Soft Tissue Surgical Support Mesh,” U.S. Pat. No. 6,669,706, entitled “Thin Soft Tissue Surgical Support Mesh,” U.S. Pat. No. 6,752,814, entitled “Devices For Minimally Invasive Pelvic Surgery,” U.S. patent application Ser. No. 10/918,123, entitled “Surgical Slings,” U.S. patent application Ser. No. 10/641,376, entitled “Spacer for Sling Delivery System,” U.S. patent application Ser. No. 10/641,192, entitled “Medical Slings,” U.S. patent application Ser. No. 10/641,170, entitled “Medical Slings,” U.S. patent application Ser. No. 10/640,838, entitled “Medical Implant,” U.S. patent application Ser. No. 10/460,112, entitled “Medical Slings,” U.S. patent application Ser. No. 10/631,364, entitled “Bioabsorbable Casing for Surgical Sling Assembly,” U.S. patent application Ser. No. 10/092,872, entitled “Medical Slings,” U.S. patent application Ser. No. 10/939,191, entitled “Devices for Minimally Invasive Pelvic Surgery,” U.S. patent application Ser. No. 10/774,842, entitled “Devices for Minimally Invasive Pelvic Surgery,” U.S. patent application Ser. No. 10/774,826, entitled “Devices for Minimally Invasive Pelvic Surgery,” U.S. patent application Ser. No. 10/015,114, entitled “Devices for Minimally Invasive Pelvic Surgery,” U.S. patent application Ser. No. 10/973,010, entitled “Systems and Methods for Sling Delivery and Placement,” U.S. patent application Ser. No. 10/957,926, entitled “Systems and Methods for Delivering a Medical Implant to an Anatomical Location in a Patient,” U.S. patent application Ser. No. 10/939,191, entitled “Devices for Minimally Invasive Pelvic Surgery,” U.S. patent application Ser. No. 10/918,123, entitled “Surgical Slings,” U.S. patent application Ser. No. 10/832,653, entitled “Systems and Methods for Sling Delivery and Placement,” U.S. patent application Ser. No. 10/642,397, entitled “Systems, Methods and Devices Relating to Delivery of Medical Implants,” U.S. patent application Ser. No. 10/642,395, entitled “Systems, Methods and Devices Relating to Delivery of Medical Implants,” U.S. patent application Ser. No. 10/642,365, entitled “Systems, Methods and Devices Relating to Delivery of Medical Implants,” U.S. patent application Ser. No. 10/641,487, entitled “Systems, Methods and Devices Relating to Delivery of Medical Implants,” U.S. patent application Ser. No. 10/094,352, entitled “System for Implanting an Implant and Method Thereof,” U.S. patent application Ser. No. 10/093,498, entitled “System for Implanting an Implant and Method Thereof,” U.S. patent application Ser. No. 10/093,450, entitled “System for Implanting an Implant and Method Thereof,” U.S. patent application Ser. No. 10/093,424, entitled “System for Implanting an Implant and Method Thereof,” U.S. patent application Ser. No. 10/093,398, entitled “System for Implanting an Implant and Method Thereof,” U.S. patent application Ser. No. 10/093,371, entitled “System for Implanting an Implant and Method Thereof,” and U.S. Provisional Patent Application Ser. No. 60/569,300, filed on May 6, 2004, the entire contents of all of which are incorporated herein by reference.

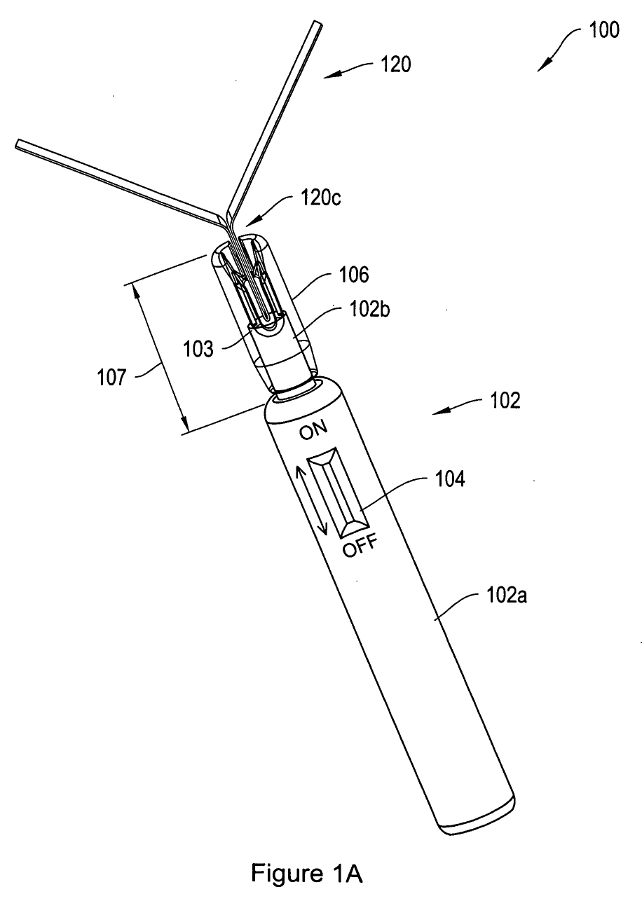

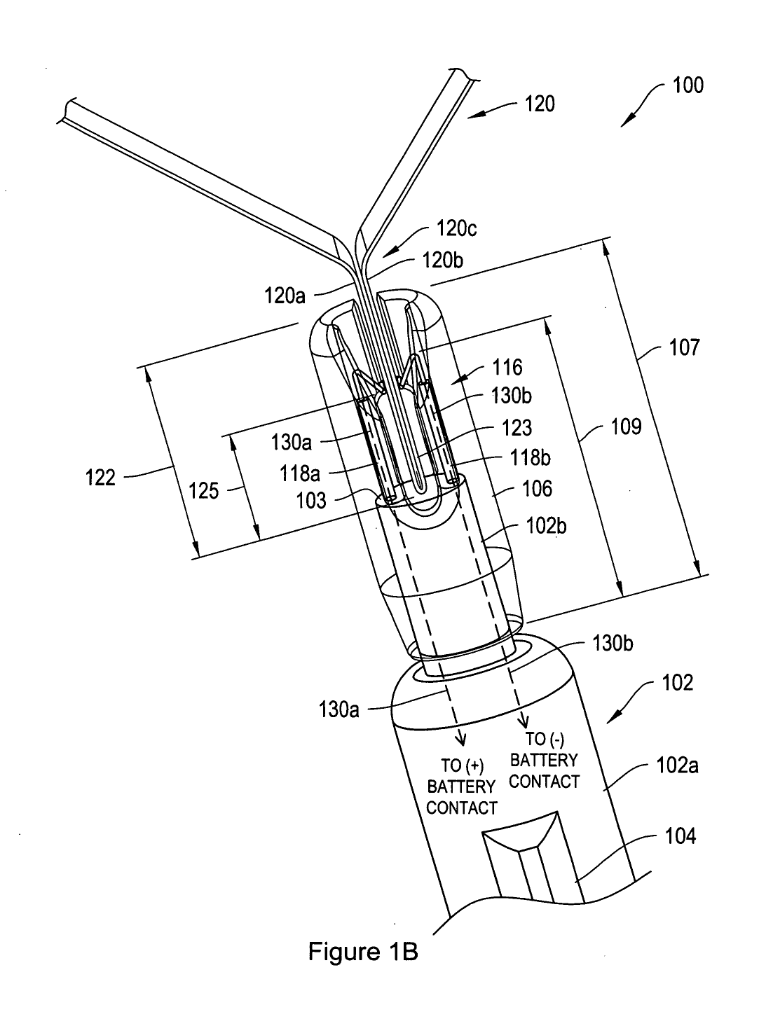

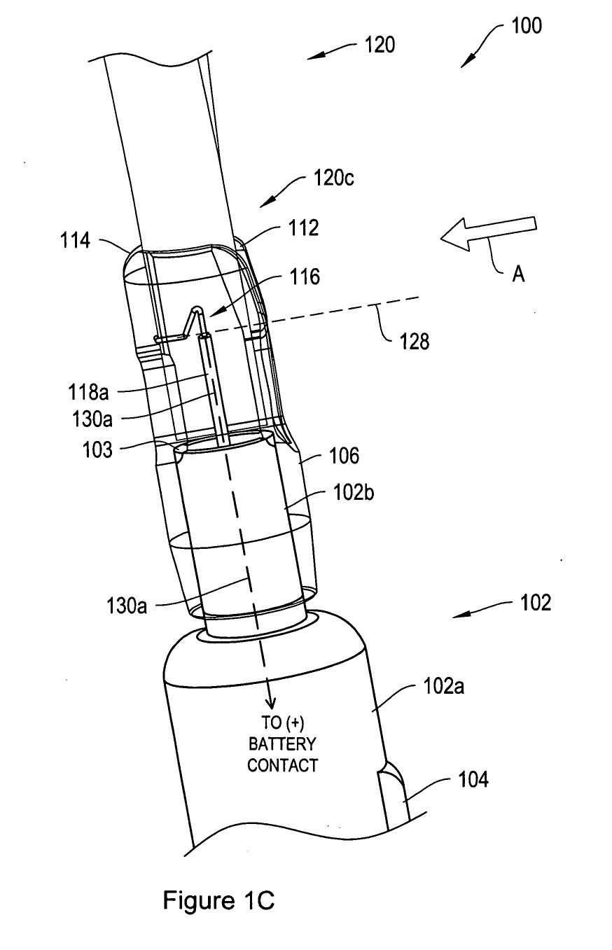

FIGS. 1A-1D show various views of a device 100 for modulating the length of an implantable sling 120, according to various embodiments of a first aspect of the invention. According to one feature, the device 100 can be used to cut through a looped portion 120c of the sling 120. According to another feature, the device 100 can be used to fuse two sling sections 120a and 120b (e.g., two legs of a looped portion 120c of a sling 120 or two separate and independent sling sections).

The device 100 includes a handle 102, a heating element 116 extending from and in interoperable interconnection with a distal portion 102b of the handle 102, and a sling guide housing 106. The sling guide housing 106 is sized and shaped to interfit over a distal portion 102b of the handle 102. Furthermore, the sling guide housing 106 is sized and shaped to guide the portion 120c of the implantable sling 120 into the sling guide housing 106 and into contact with the heating element 116. When the portion 120c of the sling 120 is guided through the sling guide housing 106, the sling 120 contacts the heating element 116 along a line 128 along the portion 120c of the sling 120. This contact line 128 is the place along the portion 120c of the sling 120 where the heating element 116 cuts through and/or fuses the sling 120. The contact line 128 is not necessarily a point of a fixed size and shape.

The device 100 includes a handle 102. The handle 102 is approximately cylindrically shaped and includes a proximal portion 102a and a distal portion 102b. Both the proximal portion 102a of the handle 102 and the distal portion 102b of the handle 102 are approximately cylindrically shaped. The distal portion 102b has a smaller outside diameter than does the proximal portion 102a. In alternative embodiments, the proximal portion 102a and the distal portion 102b can have approximately equal diameters. The heating element 116 extends from and is in interoperative interconnection with a distal most end 103 of the distal portion 102b of the handle 102. The handle 102 is about 17 cm long, although longer and shorter handle configurations are similarly contemplated. The proximal portion 102a is about 14 cm and has an outside diameter of about 1.5 cm. The distal portion 102b is about 2.5 cm long and has an outside diameter of about 1-1.5 cm. In certain embodiments, the handle includes longer or shorter proximal and/or distal portions.

The handle 102, particularly the proximal portion 102a, is used by the medical operator to hold and manipulate the device 100. The handle 102 may be composed, in whole or in part, of any of a number of materials including, but not limited to, stainless steel, plastic, a polymeric material, or other appropriate materials. The proximal portion 102a and the distal portion 102b may be composed of the same material(s) or of differing materials(s). The handle 102 may optionally include a coating. Exemplary coatings include anti-bacterial agents, anti-viral agents, and anti-fungal agents. These and other agents designed to decrease the risk of infection are especially useful when the device 100 is used during sling placement, especially when the device 100 is used within the patient or within the surgical field to modulate the length of a sling 120.

Although not specifically depicted, the device 100 may optionally include a handle grip to improve the comfort and/or ease with which the device 100 can be used by a medical operator. Exemplary grips include, but are not limited to, indentations on the surface of the handle 102, for example, indentations on the surface of the proximal portion 102a of the handle 102. A handle grip may be made from the same or different material(s) as the handle 102. In one embodiment, an area along the surface of the handle 102 composed of a material differing from that used in the remainder of the handle 102 may serve as a handle grip.

The device 100, optionally, includes a power source for supplying power to the heating element 116. In one illustrative embodiment, the power source is located in the handle 102. In one configuration, rather than including a power source, the handle 102 includes a battery compartment (such as the battery compartment of FIG. 6) sized and shaped for housing one or more batteries (e.g., one or more standard AAA, AA, or A size batteries). When the battery compartment is contained within the handle 102, one or more batteries can be added to the device 100 via a removable section of the handle 102. Use of relatively small batteries is advantageous for reducing the overall size and weight of the device 100, while still allowing use of the device 100 in the absence of an external power source.

In alternative illustrative embodiments, the device 100 is powered by an external power source. According to one feature, the device 100 includes a power supply for operable interconnection with and conditioning of the external power source. The power supply may, for example, be contained within the handle 102.

The device 100 includes a switch 104 for modulating power from the power supply/batteries/external power source (collectively “power source”) to the heating element 116. The switch 104 is substantially rectangular in shape. However, any switch shape, such as a circular/dial shape, that allows comfortable and efficient use by the medical operator may be employed. In one embodiment, the switch has only two positions, an “off” position and an “on” position. Movement of the switch 104 from the “off” position to the “on” position may occur, for example, by sliding, turning, rotating, pressing, or flipping the switch 104 from a first position to a second position. Movement of the switch 104 between the “off” and “on” positions alternatingly removes and supplies power to the heating element 116. In another embodiment, the switch 104 has more than two positions. For example, the switch 104 may have three positions, an “off” position, a low “on” position, and a high “on” position. According to one feature, the low “on” position supplies enough power to the heating element 116 for fusing, but not cutting, the sling material. According to another feature, the high “on” position supplies enough power to the heating element 116 to cut through the sling material. The switch may also include more positions or be of a continuous nature to provide a medical operator with finer control over the temperature of the heating element 116.

Regardless of the exact configuration of the switch 104, the invention contemplates several methods by which the medical operator can use the switch 104 to regulate the temperature of the heating element 116. In one embodiment, the user supplies power to the heating element 116 by moving the switch 104 from the “off” position to the “on” position a single time. The heating element 116 can then be used, for example, to cut through a section of an implantable sling, to cut through an implantable sling and fuse together the two resulting sling sections, to fuse two portions of a sling, or to fuse two separate and independent sling sections. Following achievement of the desired result, the user can move the switch 104 from the “on” position to the “off” position. In another example, the user supplies power to the heating element 116 by moving the switch 104 back and forth between the “off” and “on” positions to supply a lower level of power to the heating element 116 than that supplied by leaving the switch 104 continuously in the “on” position.

For many uses of the device 100, modulation of the amount of power supplied to the heating element 116 is not necessary. However, the invention also contemplates uses for the device 100 in which generation of multiple different power levels (e.g., modulating the temperature of the heating element 116) are advantageous. By way of example, certain sling configurations or sling materials may be more efficiently cut through and/or fused at differing temperatures. By way of further example, certain sling configurations or sling materials may be effectively cut through at one temperature but effectively fused at a different temperature. Embodiments of the device 100 that include some mechanism for modulating the temperature of the heating element 116 may allow the medical operator greater flexibility in using the device 100.

Two exemplary mechanisms for modulating the temperature of the heating element 116 are described above. According to certain other features, the device 100 may further include a temperature control element to modulate the temperature of the heating element 116. When the device 100 includes a temperature control element, the temperature control element can be incorporated into the switch 104, for example, as an additional dial, knob, or button included in the body of the switch 104. Alternatively, a temperature control element can be separate from (e.g., spaced away from) the switch 104.

Regardless of the power source or the configuration of the switch 104, the power source is put in interoperable interconnection with the heating element 116 to heat the heating element 116. As can be seen more readily in FIGS. 1B and 1C, the device 100 includes terminal portions 130a and 130b. Terminal portions 130a and 130b are in electro-communication with both the power supply and the heating element 116 via insulated portions 118a and 118b. Unlike the heating element 116, the terminal portions 130a and 130b are composed of materials that are good conductors of electricity. Thus, the terminal portions 130a and 130b do not substantially heat-up, and instead serve as a conduit through which power flows from the power source to the heating element 116. As depicted in FIG. 1B, the terminal portions 130a and 130b are in interoperative interconnection with the heating element 116 and extend through the insulator portions 118a and 118b. Terminal portions 130a and 130b continue internally through the distal portion 102b of the handle 102. When, as in the device 100, the handle 102 includes a battery compartment, the terminal portions 130a and 130b continue into the handle 102 and are in interoperative electrical interconnection with the positive and negative terminals of the battery compartment. In the depicted embodiment of FIGS. 1A and 1B, the heating element 116, including the insulator portions 118a and 118b, extends about 1 cm to about 4 cm distally from the distal most end 103 of the handle 102.

As mentioned above, the device 100 also includes a sling guide housing 106. The sling guide housing 106 has an approximately cylindrically shaped outer wall. The sling guide housing 106 is sized and shaped to interfit over the distal portion 102b of the handle 102 and to help guide a portion 120c of an implantable sling 120 into the sling guide housing 106 and into contact with the heating element 116.

The sling guide housing 106 may be made of any suitable biocompatible material, for example, a biocompatible polymeric material. In certain embodiments, all or a portion of the sling guide housing 106 may be made from substantially transparent or translucent material to enable a medical operator to view the sling joining or cutting, as the case may be, and to facilitate accurate use of the device by the medical operator.

According to the illustrative embodiment, the housing 106 is depicted as being aligned along the same axis as the handle 102. The proximate portion 102a of the handle 102 and the distal portion 102b of the handle 102 are also depicted as being aligned along the same axis. However, according to other configurations, the handle 102 and the housing 106 may be aligned along differing axes angled relative to each other, for example, to form a gun-shaped device. When the housing 106 and the handle 102 are aligned along different axes, the proximal portion 102a of the handle 102 and the distal portion 102b of the handle 102 may be aligned along the same axis or along differing axes. When the proximal portion 102a and the distal portion 102b are aligned along differing axes, either one may be aligned along the same axis as the sling guide housing 106 or along differing axes. The angle between a long axis of the handle 102 and a long axis of the sling guide housing 106 may be, for example, approximately 15°, 20°, 25°, 30°, 35°, 40°, 45°, 50°, 55°, 60°, 65°, 70°, 75°, 80°, 85°, or 90°. By way of further example, the angle between the long axis of handle 102 and the long axis of housing 106 may be approximately 95°, 100°, 105°, 110°, 115°, 120°, 125°, 130°, 135°, 140°, 145°, 150°, 155°, 160°, 165°, or 175°. Furthermore, and regardless of the angle between the long axes of any of the proximate portion 102a of the handle 102, the distal portion 102b of the handle 102, or the sling guide housing 106, any of these elements may pivot and/or rotate relative to each other.

Regardless of the alignment of the handle 102 relative to the sling guide housing 106, the invention contemplates that the sling guide housing 106 may extend and retract axially. Extending and retracting the sling guide housing 106 axially permits regulation of the distance between the heating element 116 and the distal-most end 110 of the housing 106. Extending and retracting the sling guide housing 106 axially also permits regulation of the distance between the proximate portion 102a of the handle 102 and the proximal-most end 108 of the housing 106. An axially movable sling guide housing 106 may slide smoothly and continuously in a proximo-distal direction. Alternatively, an axially movable sling guide housing 106 may move via a ratcheting mechanism. Whether the axially movable sling guide housing 106 moves via a smooth or ratcheted mechanism, the device 100 may include any conventional mechanism for locking the sling guide housing 106 in a particular position to prevent inadvertent movement of the sling guide housing 106 during device use.

As mentioned above, the sling guide housing 106 is sized and shaped to help guide the portion (illustratively a looped portion) 120c of the implantable sling 120 into the sling guide housing 106 and into contact with the heating element 116. The sling guide housing 106 is also sized and shaped for shielding the heating element 116. Shielding the heating element 116 can help prevent injury to the medical operator, and can further help prevent inadvertent contact between the heating element 116 and patient tissue. Shielding may be particularly important for embodiments of the invention in which the device is used concomitantly with sling implantation, and particularly when the device is used within the patient's body and/or within the surgical field. In embodiments in which the sling guide housing 106 is used to shield the heating element 116, it is sized and shaped to provide a barrier between the heating element 116 and the exterior surface of the sling guide housing 106. According to the illustrative embodiment of FIGS. 1A-1C, the length 107 of the sling guide housing 106 is greater than the distance 109 from the distal-most end 103 of the distal portion 102b of the handle 102 to the distal-most portion of the heating element 116. However, this need not be the case. In some embodiments, the distance 107 may be less than or equal to the distance 109. Optionally, the sling guide housing 106 is of sufficient length such that, regardless of whether the housing 106 is retracted or extended, the housing 106 continues to provide a barrier around the heating element 116.

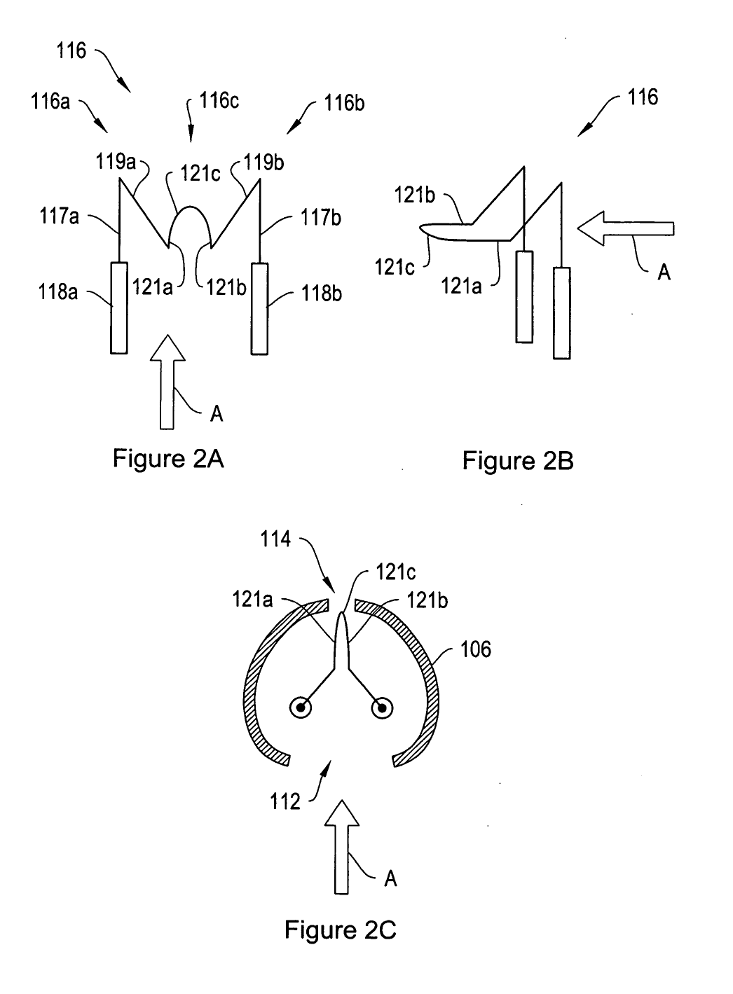

Referring to FIGS. 1A-1C and 2A-2C, the sling guide housing 106 includes two axially oriented, radially extending slots 112 and 114. As depicted, the slots 112 and 114 are diametrically opposed. Also, the slots 112 and 114 are of differing widths, though this need not be the case. Specifically, as shown in FIG. 2C, the slot 112 is wider than the slot 114. In operation, the slot 112 is the opening in the sling guide housing 106 through which the portion 120c of the implantable sling 120 initially enters the sling guide housing 106. The slot 114 is the opening through which the sling or sling sections emerge from the sling guide housing 106 following cutting through and/or fusing of the sling or sling sections.

According to other configurations of the invention, the slots 112 and 114 may be the same width. In other embodiments, the sling guide housing 106 includes one or more axially oriented, radially extending slots. Such slots may be oriented in any of a plurality of configurations relative to each other including, but not limited to, diametrically opposed to each other.

As described above, the heating element 116 is in interoperable interconnection with a power source through the terminal portions 130a and 130b via insulator portions 118a and 118b. As can be seen in FIGS. 1A, 1B, and 2A, the insulator portions 118a and 118b are spaced apart so that the sling portion 120c can enter the sling guide housing 106 through the first slot 112 and pass between the spaced apart insulator portions 118a and 118b before contacting the heating element 116.

As depicted and most readily seen in FIGS. 2A-2C, the heating element 116 includes three sections 116a, 116b, and 116c. The sections 116a and 116b are substantially V-shaped, while the section 116c is substantially U-shaped. The section 116a includes a first leg 117a extending axially in a distal direction from the insulator portion 118a. In a similar and symmetrical fashion, the section 116b includes a first leg 117b extending axially in a distal direction from the second insulator portion 118b. The section 116a also includes a second leg 119a extending proximally from a distal end of and at an angle to its first leg 117a. Thus, the first leg 117a and the second leg 119a form the substantially V-shaped section 116a. In a similar and symmetrical fashion, the section 116b includes a second leg 119b extending proximally from a distal end of and at an angle to its first leg 117b. Thus, the first leg 117b and the second leg 119b form the substantially V-shaped section 116b.

The substantially U-shaped section 116c includes first and second straight sections 121a and 121b, respectively, and a curved section 121c. The first straight section 121a extends radially outward from a proximal end of the second leg 119a of the V-shaped section 116a. In a similar and symmetrical fashion, the second straight section 121b extends radially outward from a proximal end of the second leg 119b of the V-shaped section 116b. The curved section 121c extends between the radially outward most ends of the first 121a and second 121b straight sections to complete the U-shaped section 116c.

The U-shaped section 116a of the illustrative heating element 116 is sized and shaped to cut through and/or fuse an implantable sling 120. The other sections of the heating element 116 may or may not contact the sling 120. By way of example, the other sections of the heating element 116 may be sized and shaped to help guide the section 120c of the sling 120, but may not otherwise contact the sling. Alternatively, the other sections of the heating element 116 may be sized and shaped to help guide the portion of the sling, and may incidentally contact the sling, but not for a time sufficient to cut through and/or fuse the sling. According to the illustrative embodiment, at least the U-shaped section 116c of the heating element 116 is sized and shaped to cut through and/or fuse the sling or sling sections.

According to the illustrative embodiment of FIGS. 1A-2C, the U-shaped section 116c of the heating element 116 has a length less than the width of the implantable sling 120. However, in either embodiments, the U-shaped section 116c of the heating element 116 has a length greater than or equal to the width of the implantable sling 120.

In any of the foregoing, the heating element 116 can be composed of any of a number of heat tolerant materials. For example, all or a portion of the heating element 116 can be composed of or coated with, in whole or in part, nickel chromium. Further exemplary materials include, without limitation, other nickel alloys and tungsten. Optionally, the heating element can be composed of or coated with materials that are both heat tolerant and corrosion resistant.

As shown in FIG. 1C, the diametrically opposed slots 112 and 114 are aligned relative to the apex 121c of the U-shaped section 116c of the heating element 116. In this configuration, the sling portion 120c enters the sling guide housing 106 at the first slot 112, in the direction of arrow A to contact the sling portion 120c with the heating element 116 along a contact line 128. Following contact between the heating element 116 and the sling portion 120c along the contact line point 128, the sling 120 and the device 100 are moved relative to each other along line 128 to pass the U-shaped section 116c of the heating element 116 through the sling 120 along the line 128. The sling 120 exits the sling guide housing 106 through the second slot 114. Referring to FIG. 1B, the folded length 122 of the looped sling portion 120c that enters the sling guide housing 106 is between about 2 cm and about 6 cm. (Thus, the total length is between about 4 cm and 12 cm.) Illustratively, the folded length 125 of portion 123 removed is also between about 2 cm to about 6 cm, depending on the particular geometry of the heating element 116 and the sling guide housing 106. Optionally, the U-shaped section 116c refuses the remaining sling sections 120a and 120b at the same time it cuts off the sling section 123 to form a single sling of decreased length. It should be noted that the heating element 116 and the guide housing 110 may be configured to remove any desired length of material from the sling 120.

According to another feature, the entire device 100 is optionally disposable. Such disposable devices are designed for single use, and are discarded in their entirety following the single use. Disposable, single use devices may be distributed in a sealed package labeled for individual use. Alternatively, portions of the device 100 may be disposable, while the remainder of the device 100 can be retained for subsequent use in the same patient or in a different patient. For example, the sling guide housing 106 and the heating element 116 may be removed from the handle 102 and disposed of; while the handle 102 may be retained for subsequent use. Following removal and disposal of the heating element 116 and the sling guide housing 106, the handle 102 may be sterilized prior to reuse. Alternatively, the entire device 100 can be sterilized following a first use, and subsequently used for the same patient or for a different patient.

According to the illustrative embodiment, the device 100 can be distributed in a kit along with one or more implantable slings and/or one or more sling delivery devices. In this way, the device 100 may be sold as part of a surgical kit or system for implanting a supportive sling. Such kits may be labeled with instructions for use of the device 100, and further may contain instructions for use of the one or more implantable slings and/or the one or more sling delivery devices.

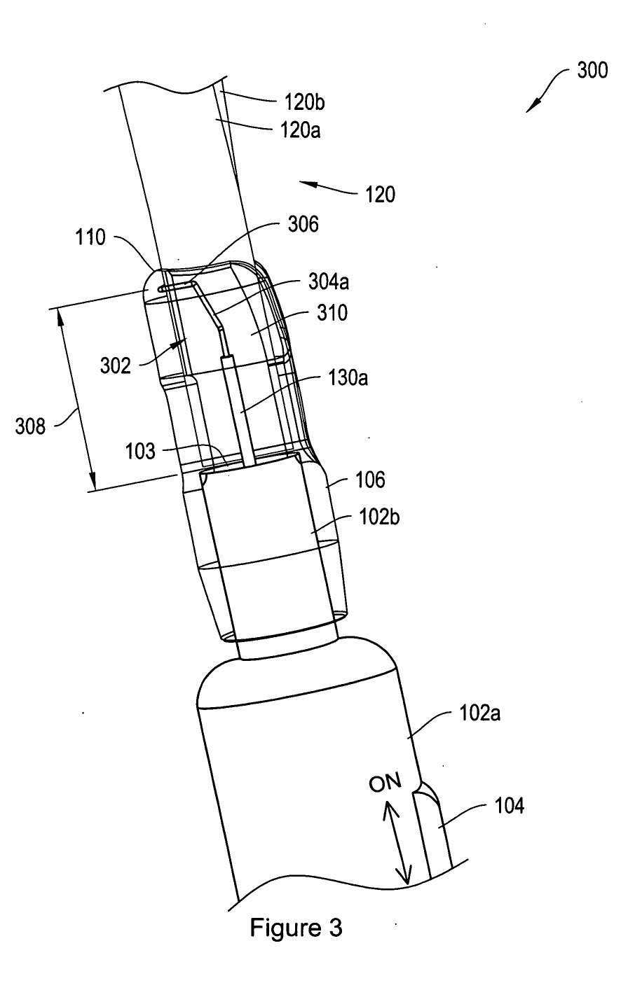

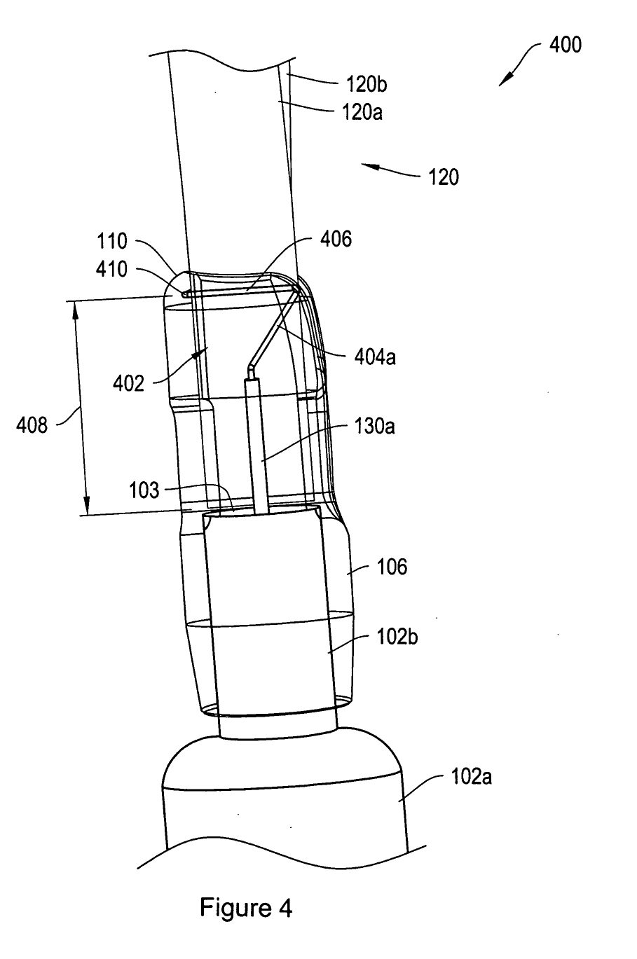

FIGS. 3 and 4 depict devices 300 and 400, respectively, which are similar to, the device 100 depicted in FIG. 1. The devices 300 and 400 differ only in the configuration of their respective heating elements 302 and 402. All other features of the devices 300 and 400 are as described above for the device 100. Additionally, the alternative embodiments contemplated and described for the device 100 are similarly contemplated for the devices 300 and 400.

More particularly, the configuration of the heating element 302 differs from the configuration of the heating element 116 of the device 100 in that rather than extending proximally at an angle, as in the case of the legs 119a and 119b of the heating element 116, the legs 304a and 304b (not visible) extend distally at an angle.

As a result of the variation in the configuration of the legs 304a and 304b of the U-shaped section 306 of the heating element 302 is positioned closer to the distal-most end 110 of the sling guide housing 106 then is the U-shaped section 116c of the heating element 116. This difference in the relative positions of the U-shaped sections of the heating elements 116 and 302 causes the length 308 of the sling portion 310 cut off by the device 300 to be longer than the length 125 of the sling portion 123 cut off by the device 100.

In FIG. 4, the heating element 402 differs from the configuration of the heating element 302 of the device 300 in that the legs 404a and 404b (not shown) angle away from the apex 410 of the U-shaped section 406, as opposed to toward it. This configuration retains the increased cutting length of the device 300, while increasing the length of the U-shaped section 406 to extend across the entire width of the sling 120.

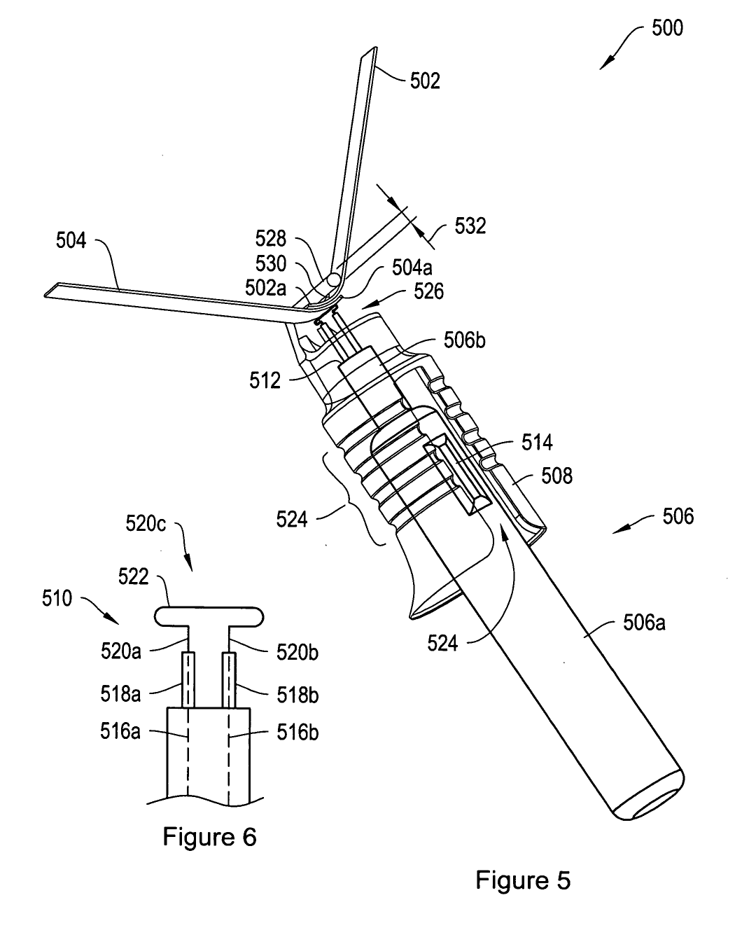

FIG. 5 shows a device 500 for fusing a first separate and independent sling section 502 with a second separate and independent sling section 504, according to another illustrative embodiment of the invention. The device 500 includes a handle 506, a heating element 508 and a sling guide housing 510.

As in the case of the previously described handles, the handle 506 includes a distal section 506b and a proximal section 506a. The proximal section 506a includes an internal battery compartment. The distal section 506b has a narrowed outside diameter as compared with the proximal section 506a. The heating element 510 extends distally from a distal most end 512 of the distal section 506b of the handle 506. A switch 514 mounts on the proximal portion 506a of the handle 506 and switches power to the heating element 510. In a similar fashion to the previously described heating elements and as shown in detail in FIG. 6, the heating element 510 connects to a power source and the switch 514 via electrically conductive terminals 516a and 516b through insulated portions 518a and 518b. The portion 520 of the heating element 510 that gets hot includes two substantially straight legs 520a and 520b and an intermediate section 520c. The two substantially straight legs 520a and 520b extend distally from the insulator sections 518a and 518b, respectively and are substantially parallel to each other. The intermediate section 520c extends between the distal ends of the legs 520a and 520b and includes a sling contacting section 522.

The sling glide housing 508 is substantially cylindrical and interfits over the distal portion 506b and onto the proximal portion 506a of the handle 506. As depicted, the sling guide housing 508 includes a finger gripping section 524. The finger gripping section 524 extends along part of the length of the proximate portion 506a of the handle 506. The finger gripping section 524 includes an axially extending slot 524, which facilitates operator access to the switch 514. Thus, the medical operator can manipulate the switch 514 while still holding the device 500 via the finger gripping section 524.

The sling guide housing 508 includes a radially oriented and radially extending notch 526 near its distal end. The notch 526 creates a spacer element 528 having a proximally facing surface 530. The heating element 510 extends into the notch 526 with the sling contacting section 522 opposing the surface 530.

In operation, the ends 502a and 504a of the sling sections 502 and 504, respectively, are overlapped and placed between the proximally facing surface 530 and the sling contacting section 522 of the heating element 510. Extending and retracting the housing 508 axially along the handle 506 regulates the distance between the sling contacting section 522 of the heating element 409 and the proximally facing surface 530 of the sling guide housing 508. The sling ends 502a and 504a are fused by sliding the sling guide housing 508 axially proximal direction along the handle 506 to sandwich the sling ends 502a and 504a between the sling contacting section 522 of the heating element 510 and the proximally facing surface 530. Extending and retracting the housing 508 axially also permits the medical operator to modulate contact between the heating element section 522 and the sling ends 502a and 504a. As in the case of the prior described embodiments, the housing 508 may slide smoothly and continuously or may move via a ratcheting mechanism. Whether the housing 508 moves via a smooth or ratcheted mechanism, the device 500 may include a mechanism for locking the housing 508 in a particular position to prevent inadvertent movement of the housing 508 during use of the device 500.

According to another feature, the spacer element 528 is sized and shaped to aid in calibrating the length of the implantable sling (e.g. the fused length of the sections 502 and 504.) Specifically, the axial thickness 532 of the spacer element 528 can be used to calibrate the space between the sling 502 and the anatomical location to be supported by the sling. This calibration can be performed in real-time as a sling implantation procedure is being performed.

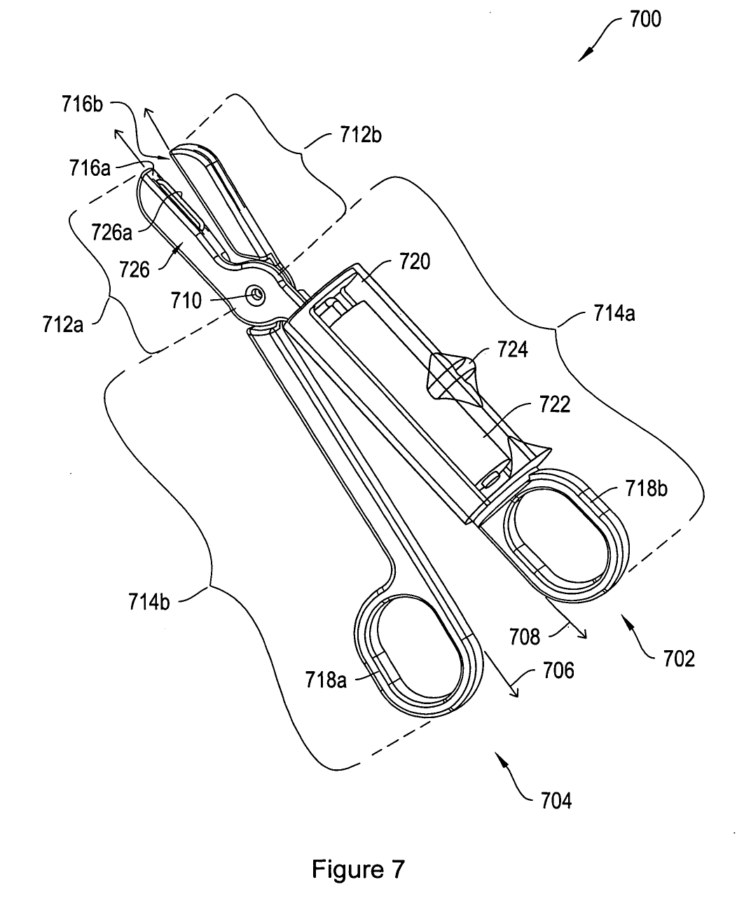

FIG. 7 shows a device 700 for cutting through and/or fusing an implantable sling, according to another illustrative embodiment of the invention. The device 700 includes a first elongated element 702 and a second elongated element 704. The first 702 and second 704 elongated elements extend along first 706 and second 708 axes, respectively, and intersect/cross at a pivot 710. Each elongated element has, with respect to the pivot 710, a proximal section 714a and 714b, and a distal section 712a and 712b, respectively. The distal sections 712a and 712b have inner surfaces 716a and 716b, respectively. Optionally, and as depicted in FIG. 7, one or both of the proximal sections includes a gripping feature 718a and 718b. Illustratively, the gripping features 718a and 718a are finger loops like those used to operate conventional scissors and forceps. However, any suitable gripping features may be employed.

According to the illustrative embodiment, each of the various sections of each of the elongated elements 702 and 704 are aligned along the same respective axis 706 and 708. However, in alternative embodiments, one of more of the sections of an elongated element, 702 or 704 may be aligned along different axes relative to each other. For example, the gripping feature 718a may be angled relative to a remainder of the proximate section 714a, and/or the gripping feature 718b may be angled relative to a remainder of the proximate section 714b. In such embodiments, the distal sections 712a and 712b may be aligned along the same or along different axes relative to the proximate sections 714a and 714b. Regardless of the angle between any of the sections of the elongated elements 702 and 704, the elements pivot relative to each other.

As depicted, the length of the distal sections 712a and 712b of each of the first and second elongated elements 702 and 704 is less than the length of the corresponding proximal sections 714a and 714b (e.g., the elements 702 and 704 intersect at a location other than their midpoint). However, in other configurations, the length of the distal sections 712a and 712b may be approximately equal to the length of the corresponding proximal sections 714a and 714b (e.g., the elements 702 and 704 intersect at approximately their midpoint). According to another configuration, the length of the distal sections 712a and 712b is greater than the length of the corresponding proximal sections 714a and 714b.

The device 700 includes a battery compartment 720 sized and shaped to house one or more batteries 722. The battery compartment 720 includes a switch 724 for switching power between the battery 722 and a heating element 726 located on the inner surface 716 of the distal section 712. It should be noted that any of the various power supplies, sources, and switches may be employed with the device 700.

As depicted, the battery compartment 720 is integrated into a portion of the first elongated element 702. Specifically, the battery compartment 720 is integrated into the proximal section 714a of the first elongated element 702. However, any suitable battery compartment in any suitable location may be utilized.

The heating element 726 extends along and is in interoperable interconnection with an inner surface 716a of the distal section 712a of the first elongated element 702. The heating element 726 is sized and shaped to cut through and/or fuse a portion of an implantable sling. An exemplary heating element 726 may have any suitable size and shape. As depicted, the heating element 726 includes a substantially straight sling contacting section 726a. However, in alternative configurations, the 726a may, for example, include curves or zig-zags. According to another configuration, heating elements such as the heating element 726, may be mounted on both inner surfaces 716a and 716b, and the device 700 may be shaped so that the heating elements come together in a substantially parallel fashion onto either side of a sling to be cut and/or fused.

The heating element 726 is in interoperable interconnection with the battery compartment 720 via electrical terminals not shown. As in prior described embodiments, the heating element 726 may be made, in whole or in part, from any of a number of heat tolerant, and optionally, corrosion resistant materials. Such materials include, without limitation, nickel chromium and tungsten.

The various configurations of the forceps-like device 700 of the invention function in substantially the same manner to cut through a portion of a sling or to fuse multiple sling sections. In the absence of power from the power source to the heating element 726, the forceps-like device 700 can be used much like a standard forceps to, for example, manipulate an implantable sling without substantially altering the structure of the sling. According to one feature, in the presence of power to the heating element 726, the heating element 726 cuts through a portion of an implantable sling to modulate the length of the implantable sling. According to another feature, in the presence of power to the heating element 726, the heating element 726 fuses two sling sections to modulate the length of the implantable sling.

Whether the device 700 is used to cut through or fuse an implantable sling, a surface of the implantable sling is brought into contact with the heating element 726 by drawing together the first 702 and second 704 elongated elements. For example, the distal portion 712a of the first elongated element 702 is brought into contact with the distal portion 712b of the second elongated element 704. In one embodiment, a portion of an implantable sling is placed between the inner surface 716a of the first elongated element 702 and the inner surface 716b of the second elongated element 704. According to certain features, the inner surface 716b of the second elongated element 704 may be sized and shaped for positioning the implantable sling with respect to the heating element 726. According to certain other features, the inner surface 716b of the second elongated element 704 may be sized and shaped to help cut through a portion of a sling or to help fuse two sling sections.

The entire device 700 is optionally disposable. Such disposable devices are designed for single use, and are discarded in their entirety following the single use. Disposable, single use devices can be distributed in a sealed package labeled for individual use. Alternatively, portions of the device 700 can be disposable while the remainder of the device 700 can be retained for subsequent use in the same patient or in a different patient. All or a portion of the device 700 may be sterilized following a first use, and subsequently used for the same patient or for a different patient.

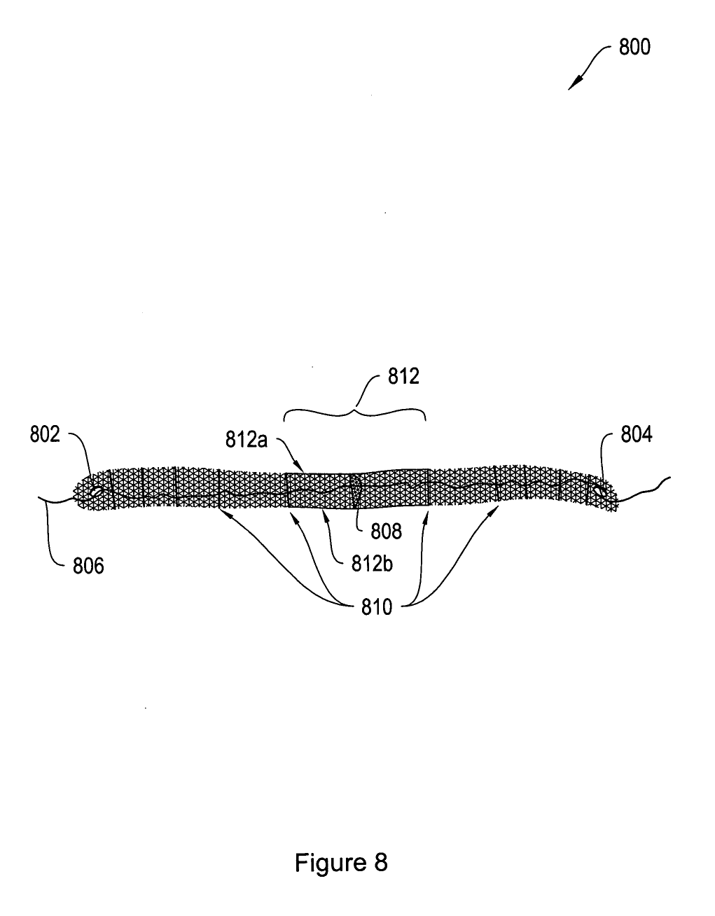

FIG. 8 depicts an implantable sling 800, according to an illustrative embodiment of the invention. As depicted, the sling 800 has two ends 802 and 804. The sling 800 also, optionally, has a backbone structure including an anti-deformation filament 806, which threads axially through the sling 800. The filament 806, alternatively, may be threaded along a portion of the length of the sling 800. The filament 806 may be formed, for example, from a suture or any other biocompatible materials. For example, the filament may be formed from a heat responsive, biocompatible material. As depicted, the illustrative filament 806 is knotted at and extends past each of the ends 802 and 804 of the sling 800. In this embodiment, the filament 806 may provide a tensioning mechanism for maintaining or adjusting tensioning of the sling 800 during and/or after implantation. As also depicted, the filament 806 is woven into the mesh of the sling 800 in a zig-zag or ragged pattern, providing a certain amount of available slack in the filament 806. In this configuration, the filament 806 enables the sling 800 to stretch to some extent (e.g., until all of the slack in the filament 806 is used), but also reduces the likelihood of the sling 800 distorting due to over stretching during implantation. The filament 806 may be left in the body of a patient, along with the sling 800 after implantation. Alternatively, in some configurations (e.g., those where the filament is not knotted to the sling ends 802 and 804), the filament 806 may be removed from the body of the patient after sling implanting.

As depicted in FIG. 8, the sling ends 802 and 804 have features distinguishing them from other portions of the sling. In such embodiments where the sling ends include useful features, for example, features to facilitate sling delivery, sling size cannot be readily modulated by cutting these free ends of the sling. To retain these useful features of the sling ends, sling size can be modulated by cutting through and/or fusing the sling at a point other than the sling ends. However, in certain embodiments, the sling ends will be otherwise indistinguishable from other portions of the sling. In such embodiments where the sling ends do not include features for sling delivery, sling size can be modulated by cutting and/or fusing the sling at any point along the sling including one or both of the sling ends.

The sling 800, optionally, includes a center-indicating feature 808 and other length- and/or position-indicating features 810 along its length. According to a preferred embodiment, the center-indicating feature 808 is distinguishable from the length-indicating features 810, for example, being differently sized and/or colored. Additionally, the length-indicating features 810 are preferably easily distinguishable from each other, for example, also being differently sized and/or colored. According to one configuration, the length-indicating features 810 are spaced at regular, known intervals. The features 808 and 810 may indicate to a medical operator the position of the sling 800 during implantation and/or enable the medical operator to adjust the length of the sling 800 prior to or during implantation. The features 810 may also be employed to indicate the effective length of the sling 800 subsequent to implantation.

According to some illustrative embodiments, the sling 800 has an initial length of about 10 to about 15 cm (about 4-6 inches) and an initial width of about 1 to about 3 cm. Slings with an initial length greater than 15 cm are similarly contemplated. Using the devices and methods exemplified throughout the application and depicted in FIGS. 1A-7, the length and/or width of the sling 800 can be modulated to customize the sling for the particular patient and indication. Given that the invention provides methods and devices for cutting through and/or fusing a sling to modulate the size of the sling, the initial length of the sling is less critical and slings of any of a number of lengths can be readily customized for the particular patient and route of delivery.

The sling 800 may initially be substantially rectangular, as illustrated in FIG. 6, or have another suitable shape. The sling 800 may have a uniform thickness over the entire length and/or width of sling 800. Alternatively, the thickness can be suitably varied at one or more locations. The thickness of the sling material may range, for example, from about 0.02 to about 0.10 cm. In one illustrative embodiment, the sling 800 is formed from a strip of mesh with any of a plurality of configurations of knits, weaves, or braids.

The sling 800 may be fabricated from any of a number of biocompatible materials, such as nylon, polyethylene, polyester, polypropylene, fluoropolymers, copolymers thereof, combinations thereof, or other suitable synthetic material(s). The material may be, for example, a synthetic material that is absorbable by the patient's body. Suitable absorbable synthetic materials can include polyglycolic acid, polylactic acid, and other suitable absorbable synthetic materials. Alternatively, the material for the sling 600 may be derived from mammalian tissue(s) or a combination of mammalian tissue(s) and synthetic material(s). The sling material may be fabricated from one or more yarns, which yarns may be made from one or more materials. The sling 800 may incorporate or be coated with one or more agents to provide a therapeutic effect, for example, to reduce discomfort, to reduce inflammation, to reduce the chance of infection, and/or to promote tissue growth. For any of the foregoing, exemplary biocompatible materials are heat responsive materials.

Referring back to FIG. 8, in one embodiment, the edge regions of the sling 800 may be configured differently depending on their intended placement in the body of the patient. For example, in one illustrative embodiment a midsection 812 of the sling 800 is located at an anatomical site, such as a midurethral or bladder neck location in the periurethral tissue, that needs to be supported. The midsection 812, in one illustrative embodiment, has smooth or rounded edges 812a and 812b, hereinafter also referred to as “non-tanged.” According to a further illustrative embodiment, other sections of the sling 800 may include tangs (e.g., sharp projections or frayed edges). Tangs are generally useful for anchoring the sling 800 and encouraging tissue growth into the sling 800. Anchoring the sling 800 in this manner generally obviates the need for additional sutures to hold the sling 800 in place. In embodiments where one or more tangs are useful, sling length can be modulated while still retaining one or more tangs. Alternatively, the number or spacing of the tangs can be altered by selectively cutting and/or fusing the sling at a portion of the sling so that one or more tangs are removed following cutting and/or fusing of the sling.

Those skilled in the art will know or be able to ascertain using no more than routine experimentation, many equivalents to the embodiments and practices described herein. Accordingly, it will be understood that the invention is not to be limited to the illustrative embodiments disclosed herein, but is to be understood from the following claims.

What is claimed is:

1. A device for cutting through an implantable sling, comprising
a handle,
a heating element in interoperative interconnection with the handle, and
a sling guide housing for guiding a portion of the implantable sling into contact with the heating element to cut through the portion of the implantable sling.

2. The device of claim 1, wherein the device includes a battery compartment.

3. The device of claim 1, wherein the device includes a switch for modulating power to the heating element.

4. The device of claim 1, wherein the device includes a temperature control element for modulating the temperature of the heating element.

5. The device of claim 1, wherein the device includes a power supply for operable interconnection with an external power source.

6. The device of claim 1, wherein the handle and the sling guide housing are aligned along the same axis.

7. The device of claim 1, wherein the handle and sling guide housing are aligned along different axes.

8. The device of claim 1, wherein the sling guide housing can be extended and retracted axially.

9. The device of claim 1, wherein the sling guide housing includes one or more slots axially oriented and extending radially through the sling guide housing.

10. The device of claim 1, wherein the sling guide housing includes two axially oriented diametrically opposed slots.

11. The device of claim 10, wherein the diametrically opposed slots are aligned with an apex of a section of the heating element.

12. The device of claim 10, wherein a width of the first slot and a width of the second slot are approximately equal.

13. The device of claim 10, wherein a width of the first slot is greater than a width of the second slot.

14. The device of claim 1, wherein the heating element comprises at least three sections and at least one section is sized and shaped to cut through the implantable sling.

15. The device of claim 14, wherein at least one section of the heating element has a length greater than or equal to the width of the implantable sling.

16. The device of claim 1, wherein the heating element comprises a nickel chromium wire.

17. A device for fusing an implantable sling, comprising
a handle,
a heating element in interoperative interconnection with the handle, and
a sling guide housing for guiding a portion of the implantable sling into contact with the heating element to fuse the portion of the implantable sling.

18. The device of claim 17, wherein the device includes a battery compartment.

19. The device of claim 17, wherein the handle and the sling guide housing are aligned along the same axis.

20. The device of claim 17, wherein the sling guide housing can be extended and retracted axially.

21. The device of claim 17, wherein the sling guide housing includes one or more slots axially oriented and extending radially through the sling guide housing.

22. The device of claim 17, wherein the sling guide housing includes two axially oriented diametrically opposed slots.

23. The device of claim 17, wherein the heating element comprises at least three sections and at least one section is sized and shaped to cut the implantable sling.

24. A device for fusing a first separate and independent implantable sling section with a second separate and independent implantable sling section, comprising
a handle,
a heating element in interoperative interconnection with the handle, and
a sling guide housing for guiding the first separate and independent implantable sling section and the second separate and independent implantable sling section into contact with the heating element, thereby fusing the first sling section with the second sling section.

25. The device of claim 24, wherein the housing includes a radially oriented extending notch near a distal end of the housing.

26. The device of claim 24, wherein a notch between a distal portion of the housing and a proximate portion of the housing forms a space between the heating element and an inner surface of the distal portion of the housing.

27. The device of claim 26, wherein the distal portion of the housing is a spacer sized and shaped to calibrate the length of the implantable sling.

28. The device of claim 24, wherein the housing can be extended and retracted axially to modulate a distance between the heating element and a distal-most end of the housing.

29. The device of claim 26, wherein the housing can be extended and retracted axially to modulate the distance between the heating element and the inner surface of the distal portion of the housing.

30. The device of claim 24, wherein the device includes a battery compartment.

31. The device of claim 24, wherein the handle and the housing are aligned along the same axis.

32. The device of claim 24, wherein the handle and housing are aligned along different axes.

33. A method for cutting through an implantable sling, comprising guiding a portion of the implantable sling into contact with the heating element of the device of claim 1, thereby cutting through the portion of the implantable sling.

34. A method for fusing an implantable sling, comprising guiding a portion of the implantable sling into contact with the heating element of the device of claim 17, thereby fusing the portion of the implantable sling.

35. A method for fusing a first separate and independent implantable sling section with a second separate and independent implantable sling section, comprising guiding the first separate and independent implantable sling section and the second separate and independent implantable sling section into contact with the heating element of claim 24, thereby fusing the first separate and independent implantable sling section with the second separate and independent implantable sling section.

36. A method for fusing a first separate and independent implantable sling section with a second separate and independent implantable sling section, comprising
contacting a first separate and independent implantable sling section and a second separate and independent implantable sling section with a heating element, and
supplying power to the heating element to fuse the first separate and independent implantable sling section with the second separate and independent implantable sling section.

37. A device for cutting through an implantable sling, comprising
a first elongated element and a second elongated element extending along first and second axes to intersect and cross at a pivot,
a heating element in operable interconnection with an inner surface of a distal portion of the first elongated element, and
a power supply in operable interconnection with the heating element.

38. The device of claim 37, wherein a proximal portion of the first elongated element includes the power supply.
